(19) United States
(12) Patent Application Publication
(10) Pub. No.: US 2013/0096092 A1
(43) Pub. Date: Apr. 18, 2013

(54) THIOETHER PRODRUG COMPOSITIONS AS ANTI-HIV AND ANTI-RETROVIRAL AGENTS

(71) Applicants: Daniel Appella, Rockville, MD (US); Ettore Appella, Chevy Chase, MD (US); John K. Inman, Bethesda, MD (US); Lisa M. Miller Jenkins, Germantown, MD (US); Ryo Hayashi, Saga (JP); Deyun Wang, Rockville, MD (US)

(72) Inventors: Daniel Appella, Rockville, MD (US); Ettore Appella, Chevy Chase, MD (US); John K. Inman, Bethesda, MD (US); Lisa M. Miller Jenkins, Germantown, MD (US); Ryo Hayashi, Saga (JP); Deyun Wang, Rockville, MD (US)

(73) Assignees: Health and Human Services, Bethesda, MD (US); The United States of America, as represented by the Secretary, Department of

(21) Appl. No.: 13/703,097

(22) PCT Filed: Jun. 10, 2011
(86) PCT No.: PCT/US11/39909

§ 371 Date: Dec. 19, 2012

Related U.S. Application Data
(60) Provisional application No. 61/353,274, filed on Jun. 10, 2010.

(51) Int. Cl. C07C 323/63; C07F 9/12; C07D 307/73; C07F 9/6571

(52) U.S. Cl. CPC C07C 323/63; C07F 9/6571; C07F 9/12; C07D 307/73
USPC 514/111; 560/251; 514/546; 560/27; 514/485; 564/154; 514/616; 558/86; 558/190; 514/119; 549/496; 514/471; 564/153

(57) ABSTRACT

Disclosed are inhibitors of retroviral growth of formula (I), that are useful in treatment of retroviral infections such as HIV. Also disclosed are a composition comprising a pharmaceutically acceptable carrier and at least one compound or salt of the invention, a method for inactivating a virus, a method for dissociating a metal ion from a zinc finger-containing protein, and a method for inhibiting the transmission of a virus.

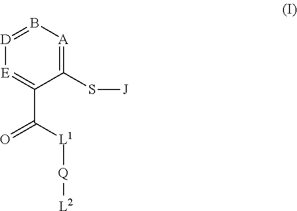

### CROSS-REFERENCE TO A RELATED APPLICATION

This patent application claims the benefit of U.S. Provisional Patent Application No. 61/353,274, filed Jun. 10, 2010, which is incorporated by reference.

### BACKGROUND OF THE INVENTION

The HIV-1 nucleocapsid protein, NCp7, is a target for the development of new antiretroviral drugs. Current combination therapies against enzymatic or viral fusion processes reduce viral replication to very low levels, but are hindered by the development of viral resistance. Thus, new therapies against other viral targets are desired to improve the therapeutic armament against HIV/AIDS.

The nucleocapsid proteins (NC) of all orthoretroviruses contain zinc fingers with the strictly conserved motif Cys-X₂-Cys-X₄-His-X₄-Cys (where X is any amino acid). The nucleocapsid of most orthoretroviruses, including HIV-1 NCp7, has two zinc fingers that comprise the primary structural elements of the protein. In addition to the zinc fingers, the surrounding residues in NCp7 are also highly conserved among the clades of HIV-1. Mutation of any of the zinc-coordinating residues renders the virus non-infectious, including conservative changes at positions that maintain the ability to coordinate zinc.

The zinc fingers of NCp7 are critical for their multiple roles during the viral replication cycle (recently reviewed in Thomas and Gorelick, Virus Res. 134, 39-63 (2008)). This protein functions as a nucleic acid chaperone, and as such, facilitates RNA-conformation-dependent reactions in the cell. Early after viral infection, NCp7 assists in tRNA annealing to genomic RNA, initiation and processivity of reverse transcription, plus- and minus-strand transfer reactions, 3′ DNA processing by integrase, and integrase-mediated strand transfer. NCp7 is encoded in the viral genome as part of the Gag polyprotein, which is specifically cleaved by the viral protease in immature virions to form the four substituent proteins: matrix (MA), capsid (CA), NCp7, and p6, as well as two spacer proteins, SP1 and SP2. As part of Gag, NCp7 is required for packaging of genomic RNA into the new virion and assists the formation of a mature dimerized double-stranded RNA genome.

While several compounds have been proposed targeting NCp7, none have advanced through clinical trials due to toxicity or lack of specificity.

In view of the foregoing, there is a desire to provide new antiretroviral compounds, in particular, compounds that target NCp7.

### BRIEF SUMMARY OF THE INVENTION

The invention provides a compound of formula (I):

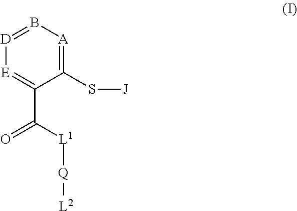

wherein A, B, D, and E are each independently selected from the group consisting of CH, N, CR⁵, CR⁶, CR⁷, and CR⁸, with the proviso that not more than two of A, B, D, and E are N,

R⁵, R⁶, R⁷, and R⁸ are independently selected from the group consisting of halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁵ and R⁶ taken together, R⁶ and R⁷ taken together, or R⁷ and R⁸ taken together form a 5- or 6-membered saturated or unsaturated ring,

L¹ is a bond or NR¹³,

L² is selected from the group consisting of hydrogen, optionally substituted alkyl, and NR¹R²,

J is selected from the group consisting of deuterium, J¹, J², J³, and J⁴,

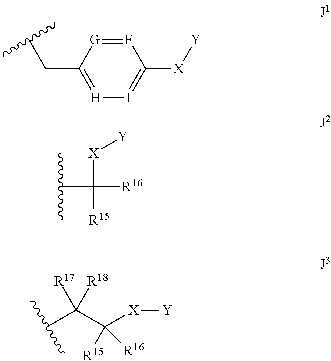

wherein J⁴ is aryl-R¹⁹ or heteroaryl-R²⁰,

wherein X is selected from the group consisting of O, S, and NR⁴,

Y is selected from the group consisting of C(O)R³, C(S)R³, C(NR⁴)R³, and P(O)(OR¹⁴)₂,

R³ is selected from the group consisting of hydrogen, optionally substituted alkyl, optionally substituted alkoxy, optionally substituted aminoalkyl, optionally substituted aryl, optionally substituted aryloxy, optionally substituted arylalkyl, optionally substituted cycloalkyl, and optionally substituted cycloalkylalkyl,

R⁴ is selected from the group consisting of H, optionally substituted alkyl, optionally substituted aryl, and optionally substituted acyl,

R¹⁴ is selected from the group consisting of H, optionally substituted alkyl, optionally substituted aryl, optionally substituted arylalkyl, optionally substituted cycloalkyl, and optionally substituted cycloalkylalkyl,

F, G, H, and I are each independently selected from the group consisting of CH, N, CR⁹, CR¹⁰, CR¹¹, and CR¹², with the proviso that not more than two of F, G, H, and I are N,

R⁹, R¹⁰, R¹¹, and R¹² are independently selected from the group consisting of halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁹ and R¹⁰ taken together, R¹⁰ and R¹¹ taken together, or R¹¹ and R¹² taken together form a 5- or 6-membered saturated or unsaturated ring,

R¹³ is selected from the group consisting of hydrogen, optionally substituted amino, optionally substituted acyl, optionally substituted aminoacyl, optionally substituted acyloxy, optionally substituted alkoxyacyl, optionally substituted aryloxyacyl, optionally substituted thioamido, hydroxy, optionally substituted alkyl, optionally substituted aryl, optionally substituted arylalkyl, optionally substituted cycloalkyl, and optionally substituted cycloalkylalkyl,

R¹⁵, R¹⁶, R¹⁷, and R¹⁸ are independently selected from the group consisting of H, optionally substituted alkyl, optionally substituted cycloalkyl, optionally substituted aryl, optionally substituted arylalkyl,

R¹⁹ and R²⁰ are selected from the group consisting of halogen, CF₃, and NO₂,

Q is selected from the group consisting of a bond, optionally substituted alkylene, optionally substituted alkylene-C(O), optionally substituted phenylene, optionally substituted phenylene-C(O), optionally substituted cycloalkylene, optionally substituted cycloalkylene-C(O), optionally substituted alkylcycloalkylene, optionally substituted alkylcycloalkylene-C(O), optionally substituted cycloalkylenealkyl, and optionally substituted cycloalkylenealkyl-C(O),

R¹ is selected from the group consisting of H, optionally substituted alkyl, optionally substituted aryl, optionally substituted heteroaryl, optionally substituted heteroalkyl, and optionally substituted heterocycloalkyl, and

R² is selected from the group consisting of H, hydroxyl, amino, optionally substituted alkyl, optionally substituted aryl, optionally substituted arylalkyl, optionally substituted alkylamine, optionally substituted arylamine, optionally substituted alkoxy, optionally substituted acyl, optionally substituted aminoacyl, optionally substituted alkoxyacyl, optionally substituted alkylthioacyl, optionally substituted arylaminoacyl, optionally substituted aryloxyacyl, optionally substituted arylthioacyl, optionally substituted heteroaryl, optionally substituted heteroalkyl, optionally substituted heterocycloalkyl and optionally substituted acylamino, or, alternatively,

R¹ and R² are optionally linked together to form an optionally substituted ring of up to about seven atoms including the N to which both are attached,

or a pharmaceutically acceptable salt thereof.

The invention also provides a pharmaceutical composition comprising a compound or salt of the invention and a pharmaceutically acceptable carrier.

The invention further provides a method for dissociating a metal ion from a zinc finger-containing protein, the method comprising contacting said zinc finger-containing protein with a compound or salt of the invention.

The invention additionally provides method for inactivating a virus, the method comprising contacting a virus with a compound or salt of the invention, whereby contacting the virus with said compound or salt inactivates the virus.

The invention also provides a method for inhibiting the transmission of a virus, the method comprising contacting a virus with a compound or salt of the invention, whereby contacting the virus with said compound inhibits the transmission thereof.

### BRIEF DESCRIPTION OF THE SEVERAL VIEWS OF THE DRAWING(S)

FIG. 1 illustrates a synthetic scheme to prepare compounds of formula (I) in accordance with an embodiment of the invention.

FIG. 2 illustrates the structures of several synthetic intermediates and compounds of formula (I) in accordance with an embodiment of the invention. Compound 7f is a comparative example.

FIG. 3 illustrates the induction of NCp7 aggregation observed for compounds in accordance with embodiments of the invention.

FIG. 4 illustrates the structures of several compounds in accordance with an embodiment of the invention.

FIG. 5 illustrates the pH stability of several compounds in accordance with an embodiment of the invention.

FIG. 6 illustrates the stability in a simulated vaginal fluid of several compounds in accordance with an embodiment of the invention.

FIG. 7 illustrates the stability in human plasma of several compounds in accordance with an embodiment of the invention.

### DETAILED DESCRIPTION OF THE INVENTION

The invention provides a compound of formula (I):

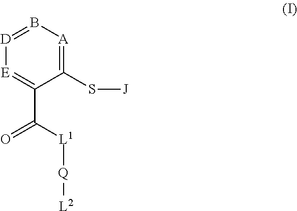

wherein A, B, D, and E are each independently selected from the group consisting of CH, N, CR⁵, CR⁶, CR⁷, and CR⁸, with the proviso that not more than two of A, B, D, and E are N,

R⁵, R⁶, R⁷, and R⁸ are independently selected from the group consisting of halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁵ and R⁶ taken together, R⁶ and R⁷ taken together, or R⁷ and R⁸ taken together form a 5- or 6-membered saturated or unsaturated ring,

L¹ is a bond or NR¹³,

L² is selected from the group consisting of hydrogen, optionally substituted alkyl, and NR¹R²,

J is selected from the group consisting of deuterium, J¹, J², J³, and J⁴,

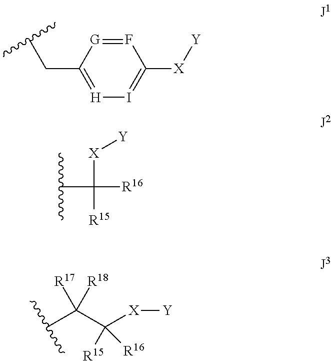

wherein J⁴ is aryl-R¹⁹ or heteroaryl-R²⁰,

wherein X is selected from the group consisting of O, S, and NR⁴,

Y is selected from the group consisting of C(O)R³, C(S)R³, C(NR⁴)R³, and P(O)(OR¹⁴)₂,

R³ is selected from the group consisting of hydrogen, optionally substituted alkyl, optionally substituted alkoxy, optionally substituted aminoalkyl, optionally substituted aryl, optionally substituted aryloxy, optionally substituted arylalkyl, optionally substituted cycloalkyl, and optionally substituted cycloalkylalkyl,

R⁴ is selected from the group consisting of H, optionally substituted alkyl, optionally substituted aryl, and optionally substituted acyl,

R¹⁴ is selected from the group consisting of H, optionally substituted alkyl, optionally substituted aryl, optionally substituted arylalkyl, optionally substituted cycloalkyl, and optionally substituted cycloalkylalkyl,

F, G, H, and I are each independently selected from the group consisting of CH, N, CR⁹, CR¹⁰, CR¹¹, and CR¹², with the proviso that not more than two of F, G, H, and I are N,

R⁹, R¹⁰, R¹¹, and R¹² are independently selected from the group consisting of halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁹ and R¹⁰ taken together, R¹⁰ and R¹¹ taken together, or R¹¹ and R¹² taken together form a 5- or 6-membered saturated or unsaturated ring,

R¹³ is selected from the group consisting of hydrogen, optionally substituted amino, optionally substituted acyl, optionally substituted aminoacyl, optionally substituted acyloxy, optionally substituted alkoxyacyl, optionally substituted aryloxyacyl, optionally substituted thioamido, hydroxy, optionally substituted alkyl, optionally substituted aryl, optionally substituted arylalkyl, optionally substituted cycloalkyl, and optionally substituted cycloalkylalkyl,

R¹⁵, R¹⁶, R¹⁷, and R¹⁸ are independently selected from the group consisting of H, optionally substituted alkyl, optionally substituted cycloalkyl, optionally substituted aryl, optionally substituted arylalkyl,

R¹⁹ is selected from the group consisting of halogen, CF₃, and NO₂,

Q is selected from the group consisting of a bond, optionally substituted alkylene, optionally substituted alkylene-C(O), optionally substituted phenylene, optionally substituted phenylene-C(O), optionally substituted cycloalkylene, optionally substituted cycloalkylene-C(O), optionally substituted alkylcycloalkylene, optionally substituted alkylcycloalkylene-C(O), optionally substituted cycloalkylenealkyl, and optionally substituted cycloalkylenealkyl-C(O),

R¹ is selected from the group consisting of H, optionally substituted alkyl, optionally substituted aryl, optionally substituted heteroaryl, optionally substituted heteroalkyl, and optionally substituted heterocycloalkyl, and

R² is selected from the group consisting of H, hydroxyl, amino, optionally substituted alkyl, optionally substituted aryl, optionally substituted arylalkyl, optionally substituted alkylamine, optionally substituted arylamine, optionally substituted alkoxy, optionally substituted acyl, optionally substituted aminoacyl, optionally substituted alkoxyacyl, optionally substituted alkylthioacyl, optionally substituted arylaminoacyl, optionally substituted aryloxyacyl, optionally substituted arylthioacyl, optionally substituted heteroaryl, optionally substituted heteroalkyl, optionally substituted heterocycloalkyl and optionally substituted acylamino, or, alternatively,

R¹ and R² are optionally linked together to form an optionally substituted ring of up to about seven atoms including the N to which both are attached,

or a pharmaceutically acceptable salt thereof.

Referring now to terminology used generically herein, the term “alkyl” means a straight-chain or branched alkyl substituent containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “alkylene,” as used herein, means a straight-chain or branched alkyl substituent containing from, for example, 1 to about 6 carbon atoms, preferably from 1 to about 4 carbon atoms, and is connected to two or more substituents at two or more different positions on the alkylene group.

The term “alkenyl,” as used herein, means a linear alkenyl substituent containing at least one carbon-carbon double bond and from, for example, about 2 to about 6 carbon atoms (branched alkenyls are about 3 to about 6 carbons atoms), preferably from about 2 to about 5 carbon atoms (branched alkenyls are preferably from about 3 to about 5 carbon atoms), more preferably from about 3 to about 4 carbon atoms. Examples of such substituents include vinyl, propenyl, isopropenyl, n-butenyl, sec-butenyl, isobutenyl, tert-butenyl, pentenyl, isopentenyl, hexenyl, and the like.

The term “alkenylene,” as used herein, means a straight-chain or branched alkenyl substituent containing from, for example, 2 to about 6 carbon atoms, preferably from 2 to about 4 carbon atoms, and is connected to two or more substituents at two or more different positions on the alkenylene group.

The term “alkynyl,” as used herein, means a linear alkynyl substituent containing at least one carbon-carbon triple bond and from, for example, 2 to about 6 carbon atoms (branched alkynyls are about 3 to about 6 carbons atoms), preferably from 2 to about 5 carbon atoms (branched alkynyls are preferably from about 3 to about 5 carbon atoms), more preferably from about 3 to about 4 carbon atoms. Examples of such substituents include ethynyl, propynyl, isopropynyl, n-butynyl, sec-butynyl, isobutynyl, tert-butynyl, pentynyl, isopentynyl, hexynyl, and the like.

The term “alkynylene,” as used herein, means a straight-chain or branched alkynyl substituent containing from, for example, 2 to about 6 carbon atoms, preferably from 2 to about 4 carbon atoms, and is connected to two or more substituents at two or more different positions on the alkynylene group.

The term “cycloalkyl,” as used herein, means a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The term “cycloalkenyl,” as used herein, means the same as the term “cycloalkyl,” however one or more double bonds are present. Examples of such substituents include cyclopentenyl and cyclohexenyl. The cyclic alkyl groups may be unsubstituted or further substituted with alkyl groups such as methyl groups, ethyl groups, and the like.

The term “heterocyclyl,” as used herein, refers to a monocyclic or bicyclic 5- or 6-membered ring system containing one or more heteroatoms selected from the group consisting of O, N, S, and combinations thereof. The heterocyclyl group can be any suitable heterocyclyl group and can be an aliphatic heterocyclyl group, an aromatic heterocyclyl group, or a combination thereof. The heterocyclyl group can be a monocyclic heterocyclyl group or a bicyclic heterocyclyl group. Suitable bicyclic heterocyclyl groups include monocylic heterocyclyl rings fused to a C₆-C₁₀ aryl ring. When the heterocyclyl group is a bicyclic heterocyclyl group, both ring systems can be aliphatic or aromatic, or one ring system can be aromatic and the other ring system can be aliphatic as in, for example, dihydrobenzofuran. Preferably, the heterocyclyl group is an aromatic heterocyclyl group. Non-limiting examples of suitable heterocyclyl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, pyridinyl, pyrimidinyl, pyrazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heterocyclyl group is optionally substituted with 1, 2, 3, 4, or 5 substituents as recited herein, wherein the optional substituent can be present at any open position on the heterocyclyl group.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2π electrons, according to Hückel's Rule.

The term “arylene” refers to an unsubstituted or substituted aromatic carbocyclic substituent as defined herein, wherein the arylene substituent is connected to two or more substituents at two or more different positions on the arylene group. For example, 1,2-dichlorobenzene can be considered to be a phenylene (arylene) group substituted with two chlorine atoms.

The term “heteroaryl,” as used herein, refers to a monocyclic or bicyclic 5- or 6-membered ring system containing one or more heteroatoms selected from the group consisting of O, N, S, and combinations thereof. The heteroaryl group can be any suitable heteroaryl. The heteroaryl group can be a monocyclic heteroaryl group or a bicyclic heteroaryl group. Suitable bicyclic heteroaryl groups include monocylic heteroaryl rings fused to a C₆-C₁₀ aryl ring. When the heteroaryl group is a bicyclic heteroaryl group, both ring systems are preferably aryl. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, pyridinyl, pyrimidinyl, pyrazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heteroaryl group is optionally substituted with 1, 2, 3, 4, or 5 substituents as recited herein, wherein the optional substituent can be present at any open position on the heteroaryl group.

The term “arylalkyl,” as used herein, refers to aryl group having an alkyl group attached thereto. The arylalkyl group can be substituted at any position of the aryl group or the alkyl group. The term “cycloalkylalkyl,” as used herein, refers to cycloalkyl group having an alkyl group attached thereto. The cycloalkylalkyl group can be substituted at any position of the cycloalkyl group or the alkyl group.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate). Similarly, the recitation of a range of 6-10 carbon atoms (e.g., C₆-C₁₀) as used with respect to any chemical group (e.g., aryl) referenced herein encompasses and specifically describes 6, 7, 8, 9, and/or 10 carbon atoms, as appropriate, as well as any sub-range thereof (e.g., 6-10 carbon atoms, 6-9 carbon atoms, 6-8 carbon atoms, 6-7 carbon atoms, 7-10 carbon atoms, 7-9 carbon atoms, 7-8 carbon atoms, 8-10 carbon atoms, and/or 8-9 carbon atoms, etc., as appropriate).

In certain embodiments, R⁵, R⁶, R⁷, and R⁸ are independently selected from the group consisting of halogen, CF₃, optionally substituted alkyl, optionally substituted alkoxy, optionally substituted aryl, NO₂, optionally substituted acylamino, optionally substituted arylamino, optionally substituted acyl, optionally substituted acyloxy, or any of R⁵ and R⁶ taken together, R⁶ and R⁷ taken together, or R⁷ and R⁸ taken together form a 5- or 6-membered saturated or unsaturated ring.

In certain preferred embodiments, R⁵, R⁶, R⁷, and R⁸ are independently selected from the group consisting of halogen, CF₃, optionally substituted alkyl, optionally substituted aryl, NO₂, optionally substituted acylamino, optionally substituted arylamino, optionally substituted acyl, or optionally substituted acyloxy.

In accordance with more preferred embodiments, R⁵, R⁶, R⁷, and R⁸ are independently selected from the group consisting of halogen, CF₃, or NO₂.

In certain embodiments, R⁹, R¹⁰, R¹¹, and R¹² are independently selected from the group consisting of halogen, CF₃, optionally substituted alkyl, optionally substituted alkoxy, optionally substituted aryl, NO₂, optionally substituted acylamino, optionally substituted arylamino, optionally substituted acyl, optionally substituted acyloxy, or any of R⁵ and R⁶ taken together, R⁶ and R⁷ taken together, or R⁷ and R⁸ taken together form a 5- or 6-membered saturated or unsaturated ring.

In certain preferred embodiments, R⁹, R¹⁰, R¹¹, and R¹² are independently selected from the group consisting of halogen, CF₃, optionally substituted alkyl, optionally substituted aryl, NO₂, optionally substituted acylamino, optionally substituted arylamino, optionally substituted acyl, or optionally substituted acyloxy.

In accordance with more preferred embodiments, R⁹, R¹⁰, R¹¹, and R¹² are independently selected from the group consisting of halogen, CF₃, or NO₂.

In certain embodiments, R¹³ is selected from the group consisting of hydrogen, optionally substituted acyl, optionally substituted acyloxy, optionally substituted alkoxyacyl, optionally substituted aryloxyacyl, optionally substituted alkyl, optionally substituted aryl, optionally substituted arylalkyl, optionally substituted cycloalkyl, and optionally substituted cycloalkylalkyl.

In accordance with more preferred embodiments, R¹³ is selected from the group consisting of hydrogen and optionally substituted alkyl.

In certain embodiments, R¹⁴ is hydrogen or optionally substituted alkyl. In more preferred embodiments, R¹⁴ is optionally substituted alkyl.

In certain preferred embodiments, R¹⁵, R¹⁶, R¹⁷, and R¹⁸ are independently selected from the group consisting of H and optionally substituted alkyl.

In certain preferred embodiments, R⁴ is selected from the group consisting of H and optionally substituted alkyl.

In certain preferred embodiments, Q is selected from the group consisting of a bond, optionally substituted alkylene-C(O), optionally substituted phenylene-C(O), optionally substituted cycloalkylene-C(O), optionally substituted alkylcycloalkylene-C(O), and optionally substituted cycloalkylenealkyl-C(O). In accordance with a more preferred embodiment, Q is optionally substituted alkylene-C(O).

In certain embodiments, R¹ is selected from the group consisting of H, optionally substituted alkyl, and optionally substituted aryl, and wherein R² is selected from the group consisting of H, optionally substituted alkyl, optionally substituted aryl, and optionally substituted arylalkyl, or, alternatively, R¹ and R² are optionally linked together to form an optionally substituted ring of up to about seven atoms including the N to which both are attached. In certain preferred embodiments, R¹ is H or optionally substituted alkyl, and optionally substituted aryl, and wherein R² is H or optionally substituted alkyl.

In an embodiment, J is deuterium.

In an embodiment, J is J¹.

In certain embodiments, J is an aromatic group substituted with one or more electron-withdrawing groups. The aromatic group may be a carbocyclic aromatic group or a heteroaryl group. Examples of suitable electron-withdrawing groups include one or more halo atoms, such as one or more fluoro atoms, one or more trifluoroalkyl groups such as trifluoromethyl groups, one or more nitro groups, and combinations thereof. In an embodiment, J is aryl-R¹⁹ or heteroaryl-R²⁰. In certain preferred embodiments, R¹⁹ and R²⁰ are NO₂. It will be understood that aryl-R¹⁹ or heteroaryl-R²⁰ encompass aryl and heteroaryl groups having two or more R¹⁹ or R²⁰ groups. In certain more preferred embodiments, J is 5-nitrofuran-2-yl.

In accordance with any of the above embodiments, L¹ is NR¹³.

In accordance with any of the above embodiments, L² is NR¹R².

In accordance with any of the above embodiments, X is O and Y is C(O)R³.

In accordance with any of the above embodiments, R³ is alkyl.

In accordance with the above embodiment, R³ is isopropyl.

In any of the above embodiments, A, B, D, and E are each independently selected from the group consisting of CH, CR⁵, CR⁶, CR⁷, and CR⁸, and F, G, H, and I are each independently selected from the group consisting of CH, CR⁹, CR¹⁰, CR¹¹, and CR¹². In accordance with certain preferred embodiments, A, B, D, and E are each CH.

In accordance with any of the above embodiments, R¹³ is hydrogen.

In certain preferred embodiments, Q is optionally substituted C₁-C₆ alkylene-C(O).

In accordance with the above preferred embodiment, Q is —CH₂CH₂C(O)— or —CH(CH₃)C(O)—.

In accordance with any of the above embodiments, R¹ and R² are both hydrogen.

In a particular embodiment, the compound is
N-{2-[4-(isopropylcarbonyloxy)benzylthio]benzoyl}-β-alaninamide or
N-{2-[4-(isopropylcarbonyloxy)benzylthio]benzoyl}-alaninamide.

In particular embodiments, the compound is selected from the group consisting of:

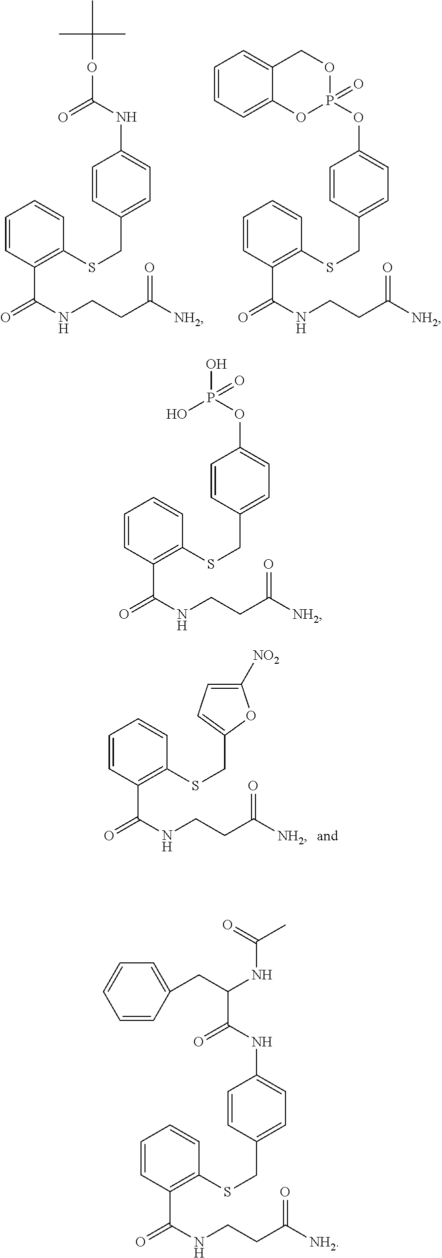

In any of the above embodiments, the compound or salt of formula (I) exists in the racemic form, in the form of its pure optical isomers, or in the form of a mixture wherein one isomer is enriched relative to the other. In particular, in accordance with the present invention, when the inventive compounds have a single asymmetric carbon atom, the inventive compounds may exist as racemates, i.e., as mixtures of equal amounts of optical isomers, i.e., equal amounts of two enantiomers. Preferably the compound or salt of formula (I) exists in the form of a single enantiomer, and more preferably in the foam of a single levorotatory enantiomer. As used herein, “single enantiomer” is intended to mean a compound that comprises more than 50% of a single enantiomer. “Single levorotatory enantiomer,” therefore, means that more than 50% of the levorotatory enantiomer is present along with less than 50% of the dextrorotatory enantiomer (this can also be referred to as a single levorotatory enantiomer), and vice versa (this can also be referred to as a single dextrorotatory enantiomer). As used herein, a levorotatory enantiomer is defined as an enantiomer having a specific rotation at a light wavelength of 589 nm that is negative. By contrast, a dextrorotatory enantiomer is defined as having a specific rotation at a light wavelength of 589 nm that is positive.

Preferably, the single enantiomer comprises at least 75% of a single enantiomer (50% enantiomeric excess) (“e.e.”), more preferably at least 90% of a single enantiomer (80% e.e.), still more preferably at least 95% of a single enantiomer (90% e.e.), even more preferably at least 97.5% of a single enantiomer (95% e.e.), and most preferably at least 99% of a single enantiomer (98% e.e.).

When the compound or salt has more than one chiral center, and can therefore exist as a mixture of diastereomers, preferably the compound or salt exists in the form of a single diastereomer. As used herein, “single diastereomer” is intended to mean a compound that comprises more than 50% of a single diastereomer.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a quinoline group or a dimethylaminoalkyl group) include hydrochloride and hydrobromide salts. The compounds of the present invention containing an acidic or basic moiety are useful in the Irwin of the free base or acid or in the form of a pharmaceutically acceptable salt thereof.

It should be recognized that the particular counter ion forming a part of any salt of this invention is usually not of a critical nature, so long as the salt as a whole is pharmacologically acceptable and as long as the counter ion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

“Contacting” refers to the act of bringing components of an interaction (e.g., a compound or salt of formula (I) with a zinc finger protein or a viral protein) or a reaction into adequate proximity such that the interaction or reaction can occur. More generally, as used herein, the term “contacting” can be used interchangeably with the following: bound to, combined with, added to, mixed with, passed over, flowed over, etc.

As used herein, “isolated,” when referring to a molecule or composition, such as, for example, a compound or salt of the present invention, a compound bound to a polypeptide, a polypeptide-RNA complex or a virus, or a compound-inactivated virus, means that the molecule or composition is separated from at least one other compound, such as a protein, other nucleic acids, etc., or other contaminants with which it is associated in vivo or in its naturally occurring state. In the context of compounds made by synthetic means, an isolated composition also includes a partially, or substantially, purified preparation containing the active ingredient. Thus, a compound, polypeptide or virion is considered isolated when it has been separated from any other component with which it is naturally associated, e.g., cell membranes, as in a cell extract, serum, and the like. An isolated composition can, however, also be substantially pure. An isolated composition can be in a homogeneous state and can be in a dry state or an aqueous solution. Purity and homogeneity can be determined, for example, using analytical chemistry techniques such as polyacrylamide gel electrophoresis (SDS-PAGE) or high performance liquid chromatography (HPLC).

As used herein, the term “nucleocapsid protein” or “NC protein” refers to the retroviral nucleocapsid protein, which is an integral part of the virion nucleocapsid where it coats the dimeric RNA genome, as described by Huang (1997) J. Virol. 71: 4378-4384; Lapadat-Tapolsky (1997) J. Mol. Biol. 268: 250-260. HIV-1's nucleocapsid protein is termed “NCp7,” see also Demene (1994) Biochemistry 33: 11707-11716.

As used herein, the term “Gag protein” or “Gag-Pol protein” refers to the polyprotein translation product of HIV-1 or other retroviruses, as described, e.g., by Fehrmann (1997) Virology 235: 352-359; Jacks (1988) Nature 331: 280-283. The “Gag protein” is processed by a viral protease to yield mature viral proteins, see, e.g., Humphrey (1997) Antimicrob. Agents & Chemotherapy 41: 1017-1023; Karacostas (1993) Virology 193: 661-671.

The term “retrovirus” as used herein refers to viruses of the Retroviridae family. These viruses can have dsRNA or ssRNA genomes transcribed by reverse transcriptase, as described by, e.g., P. K. Vogt, “Historical introduction to the general properties of retroviruses.” in Retroviruses, eds. J. M. Coffin, S. H. Hughes and H. E. Varmus, Cold Spring Harbor Laboratory Press, 1997, pp. 1-26; Murphy et al. (eds.) Archives of Virology/Supplement 10, 586 pp. (1995) Springer Verlag, Wien, N.Y. For a general description of the Retroviridae family, see the Committee on International Taxonomy of Viruses, Virology Division of the International Union of Microbiology Societies viral classifications and taxonomy. Retroviridae family members containing zinc finger motif-containing polypeptides and whose activity, e.g., replication or infectivity, can be inhibited by the compounds of the present invention include, e.g., avian sarcoma and leukosis retroviruses (alpharetroviruses), mammalian B-type retroviruses (betaretroviruses) (e.g., mouse mammary tumor virus), human T-cell leukemia and bovine leukemia retroviruses (deltaretroviruses) (e.g., human T-lymphotropic virus 1), murine leukemia-related group (gammmaretroviruses), D-type retroviruses (epsilonretroviruses (e.g., Mason-Pfizer monkey virus), and lentiviruses. Lentiviruses include, e.g., bovine, equine feline, ovine/caprine, and primate lentivirus groups, such as human immunodeficiency virus type 1 (HIV-1). Examples of particular species of viruses whose replicative capacity could be inactivated by the compounds of the present invention include HIV-1, HIV-2, SIV, BIV, EIAV, Visna, CaEV, HTLV-1, BLV, MPMV, MMTV, RSV, MuLV, FeLV, BaEV, and SSV retroviruses.

As used herein, the term “zinc finger” refers to a polypeptide motif consisting of cysteines and/or histidines that coordinate metal ions giving rise to structures involved in protein/nucleic acid and/or protein/protein interactions. The compounds or salts of the present invention are capable of modifying the structure of zinc finger peptides in such a way that allows eventual dissociation of the metal ions. Typically, the metal ion is a divalent cation, such as those of zinc or cadmium. A zinc finger motif-containing protein is commonly a highly conserved and essential structure in viruses. Zinc finger motifs are found in human papilloma virus (HPV), particularly, HPV E6 and E7 proteins (see, e.g., Ullman (1996) Biochem. J. 319: 229-239) and influenza virus (see, e.g., Nasser (1996) J. Virol. 70: 8639-8644). In most subfamilies of Retroviridae, including avian sarcoma and leukosis retroviruses, mammalian B-type retroviruses, human T-cell leukemia and bovine leukemia retroviruses, D-type retroviruses, and lentiviruses, the invariable zinc finger motif is the most highly conserved structure. Retroviral nucleocapsid, Gag and Gag-Pol proteins have zinc finger motifs. In retroviruses, the zinc finger motif typically consists of 14 amino acid residues, with four residues being invariant; one exemplary zinc finger motif is described as Cys(X)₂Cys(X)₄His(X)₄Cys and is referred to as a “CCHC zinc finger” (Henderson (1981) J. Biol. Chem. 256: 8400). Zinc fingers chelate zinc through their histidine imidazole and cysteine thiolates (Berg (1986) Science 232: 485; Bess (1992) J. Virol. 66: 840; Chance (1992) Proc. Natl. Acad. Sci. U.S.A. 89: 10041; South (1990) Adv. Inorg. Biochem. 8: 199; South (1990) Biochem. Pharmacol. 40: 123-129). CCHC zinc fingers perform essential functions in retroviral infectivity, such as packaging genomic RNA. They are also essential for early events in virus infection.

As used herein, the term “antiviral activity” means a compound has demonstrated some degree of antiviral activity in any assay, e.g., the XTT cytoprotection assay or p24 ELISA assays. As used herein, the term “virucidal” includes any degree of viral attenuation, including, but not limited to, complete inactivation or killing of a virus.

As used herein, terms such as “viral infectivity” or “index of infectivity” refer to the capacity of virus to pass from an infected cell to an uninfected cell, bringing about productive infection of the uninfected cell. For example, measurements of infectivity may be carried out by the MAGI assay, wherein the uninfected recipient cells are HeLa CD4 HIV LTR Gal cells.

As used herein, the terms “inhibit the transmission of the virus” and “antiviral activity” mean the ability of a compound to negatively affect viral replicative capacity in any way. Such inhibition of transmission, e.g., loss in replicative capacity, can be measured using any means known in the art. For example, a compound inhibits the transmission of the virus (has antiviral activity) if it diminishes a virus' ability to produce progeny, (when in the form of a virion) fuse with a cell, enter a cell, bud from a cell, survive intracellularly or extracellularly, reverse transcribe its RNA genome, translate viral proteins, process polyproteins with proteases, effect intracellular assembly of viral components into a capsid, and the like. The ability of a compound of the present invention to inhibit the transmission of a virus is not limited by any chemical or biological mechanism or pathway. A compound can inhibit infectivity or transmission (decrease replicative capacity) of a virus by, e.g.: binding to a nucleocapsid protein, such as NCp7; preventing binding of NCp7 to viral RNA or another nucleic acid; being involved in a specific chemical attack resulting in a stable or transient adduct; promoting the formation of inter- and intramolecular disulfide bonds through consequent destabilization of the NCp7 zinc finger loops; interacting with other conserved or non-conserved residues within the NCp7 protein which results in loss of function; and the like.

The present invention is further directed to a pharmaceutical composition comprising a pharmaceutically acceptable carrier and at least one compound or salt described herein.

It is preferred that the pharmaceutically acceptable carrier be one that is chemically inert to the active compounds and one that has no detrimental side effects or toxicity under the conditions of use.

The choice of carrier will be determined in part by the particular compound of the present invention chosen, as well as by the particular method used to administer the composition. Accordingly, there is a wide variety of suitable formulations of the pharmaceutical composition of the present invention. The following formulations for oral, aerosol, nasal, pulmonary, parenteral, subcutaneous, intravenous, intra-arterial, intramuscular, intraperitoneal, intrathecal, intratumoral, topical, rectal, and vaginal administration are merely exemplary and are in no way limiting.

The pharmaceutical composition can be administered parenterally, e.g., intravenously, intraarterially, subcutaneously, intradermally, or intramuscularly. Thus, the invention provides compositions for parenteral administration that comprise a solution or suspension of the inventive compound or salt dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. See, e.g., Banker and Chalmers, eds., Pharmaceutics and Pharmacy Practice, J. B. Lippincott Company, Philadelphia, pp. 238-250 (1982), and Toissel, ASHP Handbook on Injectable Drugs, 4th ed., pp. 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound or salt of the present invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compound or salt of the present invention, alone or in combination with other suitable components, can be made into aerosol formulations to be administered via inhalation. The compounds are preferably supplied in finely divided form along with a surfactant and propellant. Typical percentages of active compound are 0.01%-20% by weight, preferably 1%-10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compound or salt of the present invention may be made into suppositories by mixing with a variety of bases, such as emulsifying bases or water-soluble bases. Formulations suitable for vaginal administration may be presented as vaginal rings (i.e., intravaginal rings), pessaries, tampons, creams, gels, pastes, foams, or spray formulas containing, in addition to the active ingredient, such carriers as are known in the art to be appropriate. Desirably, formulations presented as vaginal rings provide a slow release formulation of the compound or salt within the vagina over a period of time, for example, over about one week, or over about two weeks, or over about three weeks, or over about one month. The vaginal rings can be made of any suitable material, non-limiting examples of which include silicone elastomers and ethylene-co-vinyl acetate. Non-limiting examples of vaginal rings can be found in, e.g., U.S. Pat. Nos. 4,155,991, 5,989,581, and 6,126,958, the disclosures of which are incorporated totally herein by reference. Other suitable vaginal ring products and formulations suitable for use therewith that are useful in connection with the compound or salt of the present invention will be readily apparent to those of ordinary skill in the medical and pharmaceutical arts.

It will be appreciated by one of ordinary skill in the art that, in addition to the afore described pharmaceutical compositions, the compound or salt of the present invention may be formulated as inclusion complexes, such as cyclodextrin inclusion complexes, or liposomes. Liposomes serve to target the compounds to a particular tissue, such as lymphoid tissue or cancerous hepatic cells. Liposomes can also be used to increase the half-life of the inventive compound. Liposomes useful in the present invention include emulsions, foams, micelles, insoluble monolayers, liquid crystals, phospholipid dispersions, lamellar layers and the like. In these preparations, the active agent to be delivered is incorporated as part of a liposome, alone or in conjunction with a suitable chemotherapeutic agent. Thus, liposomes filled with a desired inventive compound or salt thereof, can be directed to the site of a specific tissue type, for example hepatic cells, where the liposomes then deliver the selected compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028, and 5,019,369. For targeting to the cells of a particular tissue type, a ligand to be incorporated into the liposome can include, for example, antibodies or fragments thereof specific for cell surface determinants of the targeted tissue type. A liposome suspension containing a compound or salt of the present invention may be administered intravenously, locally, topically, etc. in a dose that varies according to the mode of administration, the agent being delivered, and the stage of disease being treated.

The invention further provides a method for dissociating a metal ion from a zinc finger-containing protein, the method comprising contacting said zinc finger-containing protein with a compound or salt of the invention. The motif can be an isolated peptide or polypeptide, or, it can be a substructure of a viral protein or a virion. The method includes contacting the zinc finger with a compound of the present invention and subsequently detecting the dissociation of the metal ion from the zinc finger protein. The cation is commonly zinc. Any methodology known in the art can be used to detect the dissociation of the metal ion. Exemplary means include, e.g., capillary electrophoresis, immune-blotting, nuclear magnetic resonance (NMR), high performance liquid chromatography (HPLC), detecting release of radioactive zinc-65, detecting fluorescence, or detecting gel mobility shift, and other techniques which would be apparent to one of skill upon review of this disclosure. These procedures can be practiced with any protocol known in the art, which are well described in the scientific and patent literature. A few exemplary techniques are set forth below.

As the invention provides a genus of novel compounds capable of dissociating a metal ion from a zinc finger in vitro, detection of the dissociation of the metal ion identifies some of the compounds within the scope of the present invention. For example, a zinc ejection assay can be used as a screen to identify compounds within the scope of the present invention. One strategy for such screening uses the XTT cytoprotection assay to monitor anti-viral activity. Alternative strategies use a Trp37 zinc ejection assay (see, e.g., U.S. Pat. No. 6,046,228) or a N-propyl gallate (NPG) fluorescence zinc ejection assay to identify compounds of the present invention that are able to act at the cellular level, e.g., on the NCp7 protein or its Gag or Gag-Pol precursors.

Certain compounds within the scope of the invention are capable of ejecting zinc from a zinc finger at some measurable rate. The rate of this effect is not necessarily indicative of potency for antiviral activity, however. Compounds which eject zinc slowly, i.e., with slow kinetics, are preferable for some uses, especially for certain in vivo applications. A “weak cation ejector” compound of the present invention would have a zinc ejection rate of about 0.86 RFU/min, or lower, as measured by the Trp37 zinc finger fluorescence assay. A “high” ejection rate would be in the range of approximately 8 RFU/min, or higher.

In certain embodiments, the zinc finger-containing protein is a viral protein. In certain embodiments, the viral protein is selected from the group consisting of a nucleocapsid protein, a Gag protein, and a Gag-Pol protein. The contacting of the protein with the compound or salt is performed in vitro or in vivo.

In certain preferred embodiments, the protein is that of a retrovirus selected from the group consisting of an HIV-1, an HIV-2, an SIV, a BIV, an EIAV, a Visna, a CaEV, an HTLV-1, a BLV, an MPMV, an MMTV, an RSV, an MuLV, a FeLV, a BaEV and an SSV retrovirus. In a preferred embodiment, the protein is that of an HIV-1 retrovirus.

The invention further provides a method for inactivating a virus, the method comprising contacting a virus with a compound or salt of the present invention, whereby contacting the virus with said compound or salt inactivates the virus. The contacting can be as described herein. The virus can be any suitable virus. Non-limiting examples of suitable viruses include an HIV-1, an HIV-2, an SIV, a BIV, an EIAV, a Visna, a CaEV, an HTLV-1, a BLV, an MPMV, an MMTV, an RSV, an MuLV, a FeLV, a BaEV and an SSV retrovirus. In certain embodiments, the virus is an HIV-1 retrovirus. In certain other embodiments, the virus is selected from the group consisting of a retrovirus, an avian sarcoma and leukosis retroviral group, a mammalian B-type retroviral group, a human T cell leukemia and bovine leukemia retroviral group, a D-type retroviral group, a murine leukemia-related group and a lentivirus group. In the above-described embodiments, preferably the contacting of the virus with the compound or salt is performed in vivo.

In some embodiments, the compound or salt of the invention is administered to inhibit the transmission of the virus. In this regard, a purpose for inhibiting the transmission of the virus refers to transmission of the virus from an infected individual to another individual. In some embodiments, inhibition of the transmission of the virus can be achieved by administration of the compound or salt intra-vaginally or intra-rectally. In certain other embodiments, inhibition of the transmission of the virus can be achieved by administration of the compound or salt parenterally, intrathecally, subcutaneously, or orally. In these embodiments, the compound or salt of the invention can be administered to a human as a pharmaceutical formulation. In other embodiments, the compound or salt of the invention can be administered to an animal as a veterinary pharmaceutical formulation.

In other embodiments of the above-described method, the method further comprises contacting the virus with another anti-retroviral agent. The anti-retroviral agent can be any suitable anti-retroviral agent. Non-limiting examples of suitable anti-retroviral agents include anti-retroviral agents selected from the group consisting of a nucleoside analogue, a nucleotide analogue, a reverse transcriptase inhibitor, an integrase inhibitor, a fusion inhibitor, an entry inhibitor, a maturation inhibitor, and a protease inhibitor. In certain preferred embodiments, the anti-retroviral agent is a nucleoside analogue which is an AZT, a ddCTP or a ddI.

In other embodiments, the contacting of the virus with the compound or salt is performed on a blood product, blood plasma, nutrient media, protein, a pharmaceutical, a cosmetic, a sperm or oocyte preparation, cells, cell cultures, bacteria, viruses, food, drink, implant or prosthesis. In these embodiments, the contacting of the virus with the compound or salt is performed in vitro.

The antiviral activity of the inventive compounds, such as viral inactivation and removal of metal ions from the zinc fingers of viral associated proteins, can be further assessed via methods disclosed in U.S. Pat. No. 7,528,274, the disclosure of which is totally incorporated herein by reference.

Without wishing to be bound by any particular theory, it is believed that the compounds of the invention undergo intracellular hydrolysis and elimination to liberate 2-mercaptobenzamides, which are then acetylated by acetyl-Co-A to produce 2-acetylmercaptobenzamides. For example, it is believed that compounds of formula (I) wherein J is J¹ undergo hydrolysis of fragment Y by esterase or by esterase followed by phosphatase to liberate the compound wherein Y is hydrogen, which then undergoes 1,4-elimination to liberate the free thiol. Compounds of formula (I) wherein J is J² undergo hydrolysis of fragment Y as described above to liberate the compound wherein Y is hydrogen, which then undergoes 1,2-elimination to liberate the free thiol. Compounds of formula (I) wherein J is J³ undergo hydrolysis of group Y as described above to liberate the compound wherein Y is hydrogen, which then undergoes elimination to liberate the free thiol. Compounds of formula (I) wherein J is J⁴ undergo bioreduction of the substituted aryl group or substituted heteroaryl group to a species which can undergo 1,4-elimination or 1,6-elimination to liberate the free thiol. The 2-acetylmercaptobenzamides are believed to specifically react with the C-terminal zinc finger of NCp7 and eject its coordinated zinc ion, irreversibly destroying protein function.

The following examples further illustrate the invention but, of course, should not be construed as in any way limiting its scope.

All chemicals and solvents were obtained from Sigma-Aldrich (Milwaukee, Wis.).

### Example 1

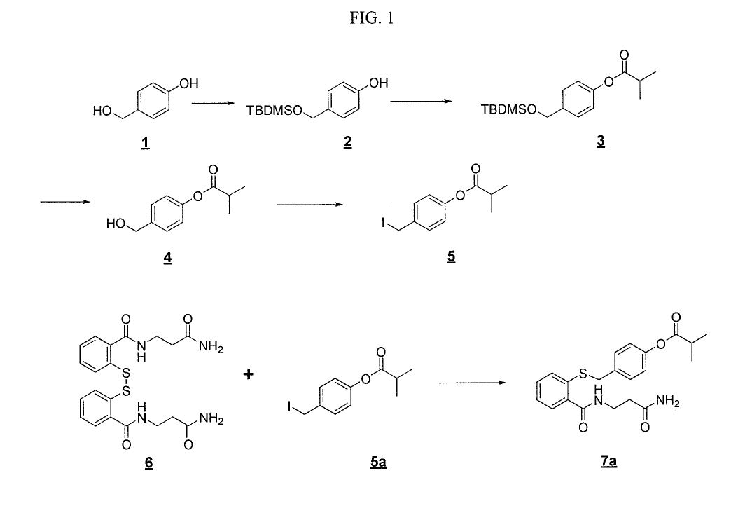

This example demonstrates the synthesis of N-{2-[4-(isopropylcarbonyl-oxy)benzylthio]benzoyl}-p-alaninamide. The reaction scheme is depicted in FIG. 1.

The synthesis of 4-(isopropylcarbonyl-oxy)benzyl iodide 5a was carried out on the basis of Iyer et al. [Bioorg. Med. Chem., 6, 1917-1922 (1996)]. 4-hydroxybenzyl alcohol 1 (20.0 mmol, 2.48 g) was dissolved in pyridine (40 ml), and the solution was cooled to 0° C. To the solution TBDMS-Cl (20.0 mmol, 3.01 g) was added at 0° C. The mixture was stirred for 3 hours at 0° C. The solvent was removed by using a rotary evaporator. EtOAc (400 ml) was added to the residue obtained. The organic layer was washed with brine and 1N hydrogen chloride (aq) and then was dried over anhydrous sodium sulfate. After filtration, the solvent was removed. The residue obtained was purified by flash chromatography (n-hexane-EtOAc=5:1) to give TBDMS-protected compound 2 (4.22 g, 89%, oil). Rf=0.36 (n-hexane-EtOAc=5:1).

TBDMS-protected compound 2 (4.22 g, 17.7 mmol) was dissolved in pyridine (40 ml). Isobutyryl chloride (1.87 ml, 17.7 mmol) was added to the solution at room temperature. After stirring for 3 hours, the solvent was removed by using a rotary evaporator. The residue was dissolved in EtOAc (400 ml), and the organic layer was washed with brine. The organic layer was dried over anhydrous sodium sulfate overnight. After removing the solvent, the residue obtained was purified by flash chromatography (n-Hx:EtOAc=9:1) giving ester compound 3 as an oil (5.26 g, 96%). Rf=0.49 (n-Hx:EtOAc=9:1).

Ester compound 3 (5.26 g, 17.1 mmol) was dissolved in anhydrous THF (20 ml). To the solution 1M TBAF solution in THF (18.8 ml, 18.8 mmol) was added dropwise to the solution at 0° C. The mixture was stirred for 1 hour at 0° C. AcOH (1.17 ml, 20.5 mmol) was added to the solution. After removing the solvent, EtOAc (400 ml) was added. The organic layer was washed with brine and dried by using anhydrous sodium sulfate. After filtration, the solvent was removed. The residue obtained was purified by flash silica gel chromatography (n-Hx:EtOAc=1:1) to give benzyl alcohol 4 as oil (3.06 g, 79%). Rf=0.55 (n-Hx:EtOAc=1:1).

Benzyl alcohol 4 (2.46 g, 12.7 mmol) was dissolved in DCM (10 ml). After addition of imidazole (1.30 g, 19.1 mmol) and iodine (6.45 g, 25.4 mmol), triphenylphosphine (5.00 g, 19.1 mmol) solution in THF (10 ml) was added dropwise to the solution at 0° C. The mixture was stirred for 2 hours at room temperature. The solvent was removed by using a rotary evaporator. The residue obtained was purified by short flash chromatography (n-Hx; EtOAc=5:1) to obtain benzyl iodide 5a as an oil (3.69 g, 96%). After purification, benzyl iodide 5a was used for the next reaction immediately. Rf=0.68 (n-Hx; EtOAc=5:1).

Disulfide 6 was synthesized as previous reported [Bioorg. Med. Chem., 14, 6437-6450 (2004)]. Thioether 5a was synthesized by utilizing a reaction Tang et al. reported [Synthesis 2007, No. 1, 0085-0091]. Disulfide 6 (2.70 g, 6.05 mmol), benzyl iodide 5a (3.69 g, 12.1 mmol), and potassium carbonate (3.34 g, 24.2 mmol) were suspended in DMF (20 ml). To the suspension sodium hydroxymethanesulfinate hydrate (4.29 g, 36.3 mmol) was added, followed by water (ca. 100 μl) at room temperature. The mixture was stirred overnight, resulting in a clear orange solution. DCM (200 ml) was added, and then the organic layer was washed with brine and 1N HCl aq. The organic layer was dried over anhydrous sodium sulfate. After filtration and evaporation, white solid was obtained. The white solid was recrystallized from EtOAc to give thioether 7a (3.39 g, 70%). ¹H-NMR (400 MHz, CDCl₃): δ 7.49 (1H, m), 7.40 (1H, m), 7.34 (2H, m) 7.18 (2H, m), 6.93 (2H, m), 5.83 (1H, boad), 5.25 (1H, broad), 4.06 (2H, s), 3.67 (2H, m), 2.53 (2H, t), 1.30 (6H, d). MALDI-TOF MS: calcd for C₂₁H₂₄N₂O₄S [M+Na]+423.48, [M+K]+439.59. found 424.19, 440.21.

### Example 2

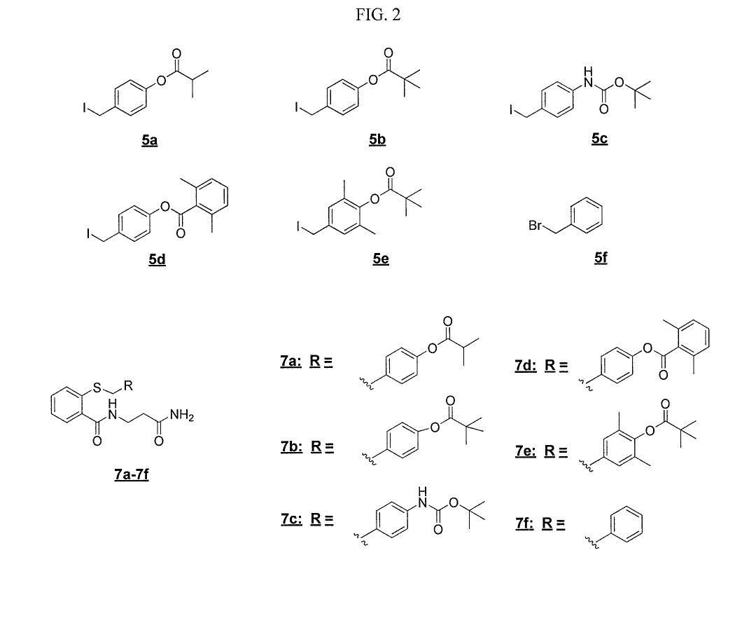

This example demonstrates a general synthesis for the preparation of the compounds of the invention. The structures of intermediates 5a-5e and of products 7a-7f are depicted in FIG. 2.

Sodium borohydride (9.0 mg, 0.22 mmole) was added to a solution of disulfide compound 6 (100 mg, 0.22 mmole) in 3 mL of dry DMF. The reaction mixture was stirred at room temperature under nitrogen for 1 h. Benzyl iodide/bromide derivatives 5a-5f or benzyl bromide (0.44 mmole) were then added to the above reaction mixture and stirred for another 4 h under nitrogen. The solution was concentrated and the residue was purified with flash chromatography (dichloromethane: methanol, 95:5) to afford final products 7a-7f.

### Example 3

The following compounds were prepared in accordance with the method described in Example 2. The analytical data is set forth herein.

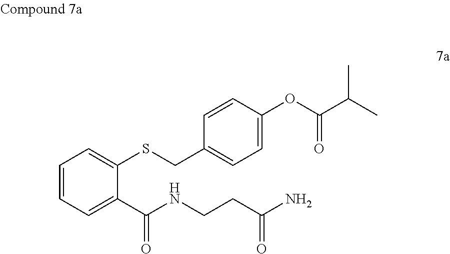 [SMILES: CC(C)C(=O)Oc1ccc(CSc2ccccc2C(=O)NCCC(N)=O)cc1]

White solid (102 mg, 65% yield). ¹H NMR (400 MHz, DMSO) δ 8.31 (t, J=5.6, 1H), 7.50-7.29 (m, 6H), 7.20 (t, J=7.0, 1H), 7.05 (d, J=8.5, 2H), 6.85 (s, 1H), 4.19 (s, 2H), 3.39 (dd, J=7.1, 13.1, 2H), 2.80 (dt, J=7.0, 13.9, 1H), 2.35 (t, J=7.3, 2H), 1.22 (d, J=7.0, 6H); ¹³C NMR (100 MHz, DMSO) δ 175.51, 172.92, 168.00, 149.99, 136.95, 135.95, 135.18, 130.49, 130.36, 128.30, 128.14, 125.50, 122.12, 36.39, 36.27, 35.37, 33.76, 19.13; HRMS m/z for C₂₁H₂₅N₂O₄S [M+H]⁺, calcd 401.1457. found 401.1523.

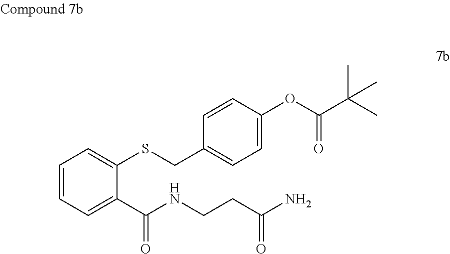 [SMILES: CC(C)(C)C(=O)Oc1ccc(CSc2ccccc2C(=O)NCCC(N)=O)cc1]

White solid (134 mg, 72% yield). ¹H NMR (400 MHz, DMSO) δ 8.31 (t, J=5.6, 1H), 7.48-7.30 (m, 6H), 7.21 (t, J=7.4, 1H), 7.03 (d, J=8.4, 2H), 6.86 (s, 1H), 4.20 (s, 2H), 145-3.37 (dt, J=7.0, 13.9, 2H), 2.36 (t, J=7.3, 2H), 1.29 (s, 9H); ¹³C NMR (100 MHz, DMSO) δ 176.87, 172.95, 168.02, 150.18, 137.05, 135.89, 135.19, 130.48, 130.35, 128.40, 128.14, 125.53, 122.07, 71.76, 36.46, 36.29, 35.37, 27.22; HRMS m/z for C₂₂H₂₇N₂O₄S [M+H]⁺, calcd 415.1613. found 415.1689.

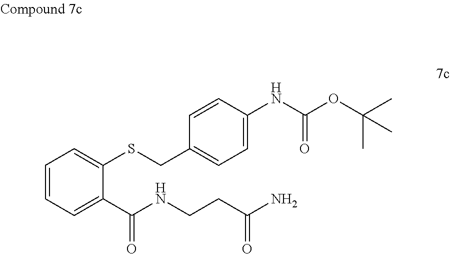 [SMILES: CC(C)(C)OC(=O)Nc1ccc(CSc2ccccc2C(=O)NCCC(N)=O)cc1]

White solid (150 mg, 78% yield). ¹H NMR (400 MHz, DMSO) δ 9.32 (s, 1H), 8.28 (s, 1H), 7.38 (m, 6H), 7.24 (d, J=8.3, 2H), 7.19 (t, J=7.3, 1H), 6.84 (s, 1H), 4.10 (s, 2H), 3.37 (dd, J=6.9, 13.2, 2H), 2.33 (t, J=7.3, 2H), 1.47 (s, 9H); ¹³C NMR (100 MHz, DMSO) δ 172.92, 168.02, 153.22, 138.93, 136.88, 136.28, 130.84, 130.31, 129.75, 128.26, 128.07, 125.35, 118.53, 79.47, 36.74, 36.27, 35.37, 28.59; HRMS m/z for C₂₂H₂₈N₃O₄S [M+H]⁺, calcd 430.1722. found 430.1792.

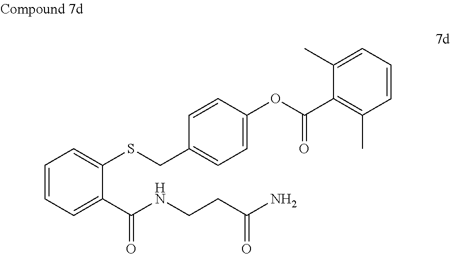 [SMILES: Cc1cccc(C)c1C(=O)Oc1ccc(CSc2ccccc2C(=O)NCCC(N)=O)cc1]

White solid (140 mg, 68% yield). ¹H NMR (400 MHz, DMSO) δ 8.32 (t, J=5.6, 1H), 7.50 (d, J=8.5, 2H), 7.46 (d, J=8.1, 1H), 7.36 (m, 4H), 7.29-7.22 (m, 3H), 7.19 (t, J=7.2, 2H), 6.85 (s, 1H), 4.24 (s, 2H), 3.39 (dd, J=7.2, 13.1, 2H), 2.40 (s, 6H), 2.35 (t, J=7.3, 2H); ¹³C NMR (100 MHz, DMSO) δ 172.91, 168.13, 168.00, 149.57, 136.94, 135.93, 135.88, 135.12, 133.04, 130.77, 130.49, 130.39, 128.24, 128.21, 128.16, 125.50, 122.19, 100.00, 36.29, 35.37, 19.81. HRMS m/z for C₂₆H₂₇N₂O₄S [M+H]⁺, calcd 463.1613. found 463.1687.

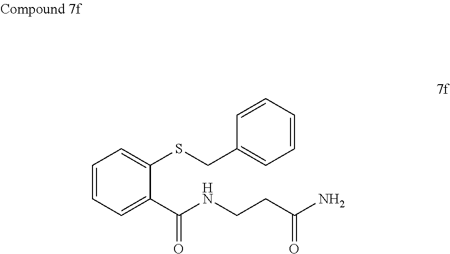 [SMILES: NC(=O)CCNC(=O)c1ccccc1SCc1ccccc1]

White solid (106 mg, 75% yield). ¹H NMR (400 MHz, DMSO) δ 8.30 (t, J=5.5, 1H), 7.49-7.29 (m, 8H), 7.25 (d, J=7.2, 1H), 7.20 (t, J=5.4, 1H), 6.85 (s, 1H), 4.18 (s, 2H), 3.38 (dd, J=7.1, 13.2, 2H), 2.34 (t, J=7.4, 2H); ¹³C NMR (100 MHz, CD₃OD) δ 176.62, 171.62, 139.26, 138.82, 135.98, 131.94, 131.43, 130.19, 129.58, 128.90, 128.33, 127.43, 39.97, 37.42, 36.01; HRMS m/z for C₁₇H₁₀N₂O₂S [M+H]⁺, calcd 315.1089. found 315.1177.

### Example 4

This example illustrates the cytoprotection and virucidal activity observed for a compound in accordance with an embodiment of the invention.

The HIV cytoprotection assay has been previously described in Weislow, O, S. et al., J. Natl. Cancer Inst. 81, 577-86 (1989); Rice, W. G. et al., Antimicrob. Agents Chemother. 41, 419-26 (1997), and Rice, W. G. et al., Proc. Natl. Acad. Sci. USA 90, 9721-4 (1993). CEM-SS cells infected with HIV-1_{IIIB} or monocyte-macrophages infected with HIV_{BaL} were incubated with the compound for 6 days and antiviral activity determined by measurement of cell survival (cytoprotection) using XTT (2,3-bis(2-methoxy-4-nitro-5-sulfophenyl)-5-[(phenylamino)carbonyl]-2H-tetrazolium hydroxide) dye reduction. AZT was used as a positive control for all assays with each assay performed in duplicate with triplicate determinations per condition. Compound toxicity was measured the same way in uninfected CEM-SS cells. Effective antiviral concentrations providing 50% cytoprotection (EC₅₀) and cellular growth inhibitory concentrations causing 50% cytotoxicity (IC₅₀) were calculated.

Cell-free virus inactivation assays were performed as previously described in Song, Y. et al., Bioorg. Med. Chem. 10, 1263-73 (2002). Briefly, HIV-1_{IIIB} was incubated with three concentrations of each compound for 2 h at 37° C. and then pelleted at 4° C. for 90 minutes at 18,000×g. Following several washes with DPBS to remove unbound compound, the virus was resuspended in cell culture media and added to CEM-SS cells at a titer to yield 85-90% cell killing at day 6 post-infection.

Three compounds were assayed for antiviral activity using the XTT cytoprotection assay in CEM-SS cells infected with HIV-1_{IIIB} and in monocyte-macrophages infected with HIV_{BaL} and assayed for virucidal activity. The compounds are compound 7a (invention), 8 (comparative), and 7f (comparative). The structure of compound 8 is as follows:

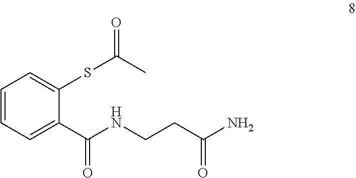 [SMILES: CC(=O)Sc1ccccc1C(=O)NCCC(N)=O]

The results are set forth in Table 1.

TABLE 1

| | Compound | 8 | 7a | 7f |
|---|---|---|---|---|
| CEM-SS cells | EC₅₀ (μM) | 0.6 | 0.2 | 34.9 |
| | TC₅₀ (μM) | >100 | >100 | >100 |
| Monocytes-macrophages | EC₅₀ (μM) | 2.0 | 0.6 | >100 |
| | TC₅₀ (μM) | >100 | >100 | >100 |
| Virucidal activity | EC₅₀ (μM) | 0.9 | 9.8 | >100 |

As is apparent from the results set forth in Table 1, compound 7a exhibited approximately 3 times lower EC₅₀ in the cytoprotection assay against infected CEM-SS cells, an approximately 3.3 times lower EC₅₀ in the cytoprotection assay against infected monocytes-macrophages, and an approximately 10 times greater EC₅₀ in the antiviral assay against HIV-1_{IIIB} than exhibited by thiol ester 8. Benzyl thioether 7f exhibited an approximately 175 times greater EC₅₀ in the cytoprotection assay against infected CEM-SS cells, and was inactive in the cytoprotection assay against infected monocytes-macrophages and in the antiviral assay against HIV-1_{IIIB} as compared to compound 7a.

### Example 5

This example illustrates the induction of NCp7 aggregation observed for compounds in accordance with embodiments of the invention.

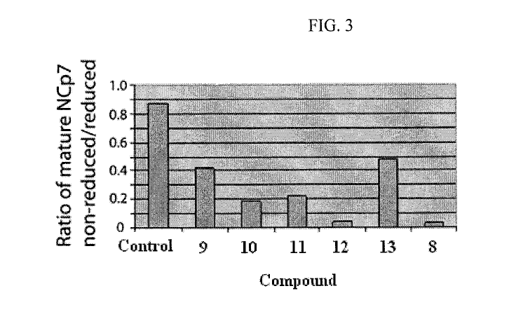

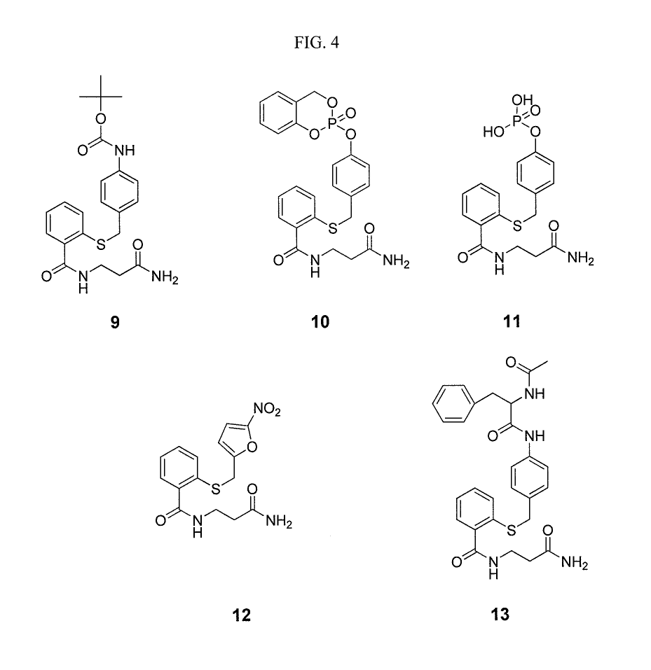

Compounds were tested for their ability to cause NCp7 aggregation in H9 cells chronicalll infected with HIV-1 as described in Miller Jenkins et al., Nat. Chem. Biol., 6: 887-9 (2010). NCp7 was separated on both reducing and non-reducing gels and quantitated. Compounds 9-13 are inventive. Compound 8 is a positive control. The structures of compounds 9-13 are depicted in FIG. 4. The control assay was performed in the absence of added compound. The results are illustrated in FIG. 3.

As is apparent from the results depicted in FIG. 3, compounds 9-13 as well as positive control compound 8 produced a decrease in the ratio of NCp7 in non-reducing versus reducing conditions, due to the increased presence of crosslinked material and, thus, increased activity of the compounds.

### Example 6

This example illustrates the cytoprotection and virucidal activity observed for four compounds in accordance with embodiments of the invention.

Four compounds along with a positive control were assayed for antiviral activity using the XTT cytoprotection assay as described in Example 4. The cells used were CEM-SS cells infected with HIV-1_{RF}, monocyte-macrophages infected with HIV_{BaL}, and peripheral blood mononuclear cells (PBMC) infected with HIV-1_{HT/92/599}. The compounds are compound 8 (comparative), 7a (invention), 10 (invention), 11 (invention), and 12 (invention). The results are set forth in Table 2.

TABLE 2

| | Compound | 8 | 7a | 10 | 11 | 12 |
|---|---|---|---|---|---|---|
| CEM-SS cells | EC₅₀ (μM) | 0.6 | 0.2 | 0.89 | 3.28 | 0.85 |
| | TC₅₀ (μM) | >100 | >100 | >50 | >50 | 8.24 |
| PBMC | EC₅₀ (μM) | 5.74 | 4.06 | 20.4 | ND | 8.15 |
| | TC₅₀ (μM) | >100 | >100 | >50 | ND | >50 |
| Monocyte/ | EC₅₀ (μM) | 1.97 | 0.62 | 0.18 | ND | 1.61 |
| Macrophage | TC₅₀ (μM) | >100 | >100 | >50 | ND | >50 |

As is apparent from the results set forth in Table 2, the compounds generally showed low cytotoxicity, although compound 12 was somewhat toxic in CEM-SS cells. As this compound showed no toxicity in primary cells, most likely this toxicity is unique to these transformed cells. In addition, all of the compounds showed similar activity in the different cell types, although 12 was more active the in PBMCs and 7 in the monocyte/macrophage cells. The activity was comparable to the thioester control compound 8.

### Example 7

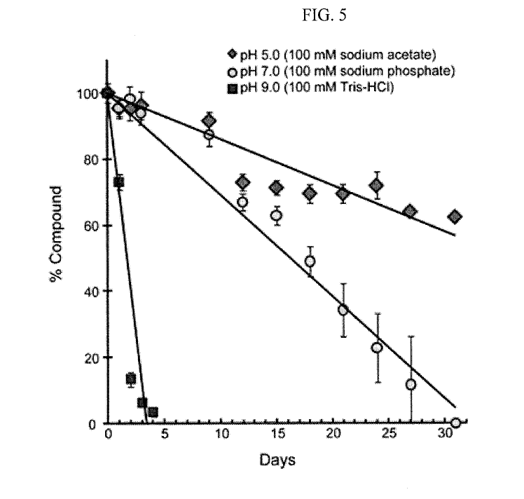

This example illustrates the pH stability of compound 7a. Compound 7a was dissolved in DMSO and diluted into the 100 mM sodium acetate buffer at pH 5.0 (shown in FIG. 5 as diamonds), 100 mM sodium phosphate buffer at pH 7.0 (shown in FIG. 5 as circles), and 100 mM Tris-HCl buffer at pH 9.0 (shown in FIG. 5 as squares) at a final compound concentration of 250 μM. The samples were incubated at 25° C. over the course of 30 days and the amount of intact compound was measured over time using RP-HPLC. The results are illustrated in FIG. 5.

As is apparent from the results depicted in FIG. 5, compound 7a was most stable at acidic pH (i.e., pH 5.0).

### Example 8

This example illustrates the stability in a vaginal fluid stimulant observed for three compounds in accordance with embodiments of the invention as well as a control compound.

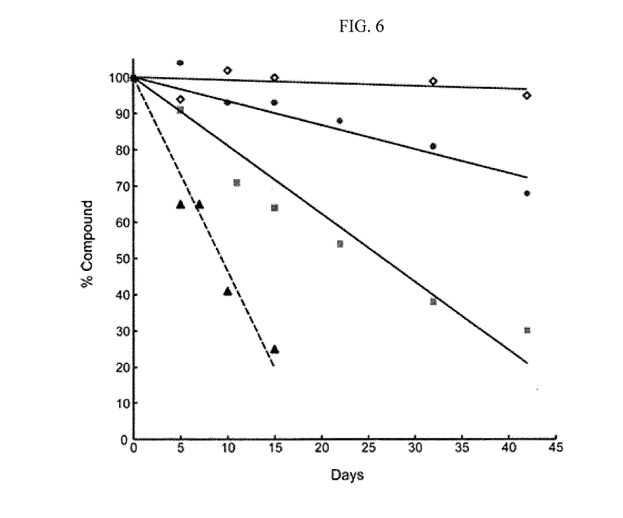

Compound 8 (comparative), compound 7f (comparative), compound 7a (invention), and compound 9 (invention) were dissolved in DMSO and diluted into the vaginal fluid simulant described by Owen and Katz, Contraception, 59: 91-5 (1999). The final concentration of the compounds was 400 μM. The samples were incubated at 25° C. over the course of 30 days and the amount of intact compound was measured over time using RP-HPLC. Compound 8 is shown as triangles, compound 7a is shown as circles, compound 9 is shown as squares, and compound 7f is shown as diamonds. The results are illustrated in FIG. 6.

As is apparent from the results depicted in FIG. 6, compound 7a was significantly more stable in a vaginal fluid simulant than compound 8.

### Example 9

This example illustrates the human plasma stability of some of the compounds in accordance with embodiments of the invention.

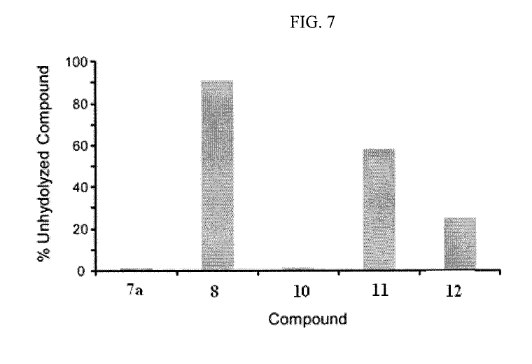

Compound 8 (comparative), compound 7a (invention), compound 10 (invention), compound 11 (invention), and compound 12 (invention) were dissolved in DMSO and diluted into 50% human plasma in PBS buffer at a final concentration of 20 μM. The samples were incubated at 37° C. for 1 hour and the amount of intact compound measured using RP-HPLC. The results are illustrated in FIG. 7.

As is apparent from the results depicted in FIG. 7, compounds 7a and 10 did not exhibit stability in human plasma, whereas compounds 11 and 12 exhibited improved stability. Thus, embodiments of the invention may be useful systemically.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 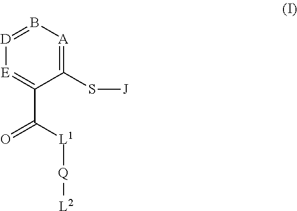
wherein A, B, D, and E are each independently selected from CH, N, CR⁵, CR⁶, CR⁷, and CR⁸, with the proviso that not more than two of A, B, D, and E are N,
R⁵, R⁶, R⁷, and R⁸ are independently selected from halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁵ and R⁶ taken together, R⁶ and R⁷ taken together, or R⁷ and R⁸ taken together form a 5- or 6-membered saturated or unsaturated ring,
L¹ is a bond or NR¹³,
L² is selected from H, optionally substituted alkyl, and NR¹R²,
J is selected from deuterium, J¹, J², J³, and J⁴, 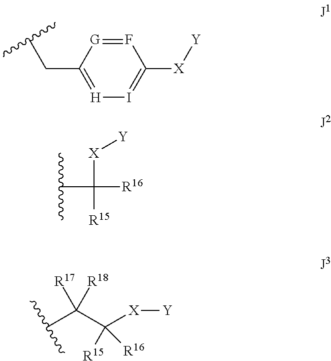
wherein J⁴ is aryl-R¹⁹ or heteroaryl-R²⁰,
wherein X is selected from O, S, and NR⁴,
Y is selected from C(O)R³, C(S)R³, C(NR⁴)R³, and P(O)(OR¹⁴)₂,
R³ is selected from hydrogen, optionally substituted alkyl, optionally substituted alkoxy, optionally substituted aminoalkyl, optionally substituted aryl, optionally substituted aryloxy, optionally substituted arylalkyl, optionally substituted cycloalkyl, and optionally substituted cycloalkylalkyl,
R⁴ is selected from H, optionally substituted alkyl, optionally substituted aryl, and optionally substituted acyl,
R¹⁴ is selected from H, optionally substituted alkyl, optionally substituted aryl, optionally substituted arylalkyl, optionally substituted cycloalkyl, and optionally substituted cycloalkylalkyl, or, taken together, form a 5- or 6-membered ring,
F, G, H, and I are each independently selected from CH, N, CR⁹, CR¹⁰, CR¹¹, and CR¹², with the proviso that not more than two of F, G, H, and I are N,
R⁹, R¹⁰, and R¹² are independently selected from halogen, CF₃, optionally substituted alkyl, optionally substituted alkoxy, optionally substituted aryl, NO₂, optionally substituted alkylthio, optionally substituted amino, optionally substituted acylamino, optionally substituted arylamino, optionally substituted acylthio, optionally substituted acyl, optionally substituted acyloxy, hydroxy, mercapto, and optionally substituted thioamido, or any of R⁹ and R¹⁰ taken together, R¹⁰ and R¹¹ taken together, or R¹¹ and R¹² taken together form a 5- or 6-membered saturated or unsaturated ring,
R¹³ is selected from hydrogen, optionally substituted amino, optionally substituted acyl, optionally substituted aminoacyl, optionally substituted acyloxy, optionally substituted alkoxyacyl, optionally substituted aryloxyacyl, optionally substituted thioamido, hydroxy, optionally substituted alkyl, optionally substituted aryl, optionally substituted arylalkyl, optionally substituted cycloalkyl, and optionally substituted cycloalkylalkyl,
R¹⁵, R¹⁶, R¹⁷, and R¹⁸ are independently selected from H, optionally substituted alkyl, optionally substituted cycloalkyl, optionally substituted aryl, optionally substituted arylalkyl,
R¹⁹ and R²⁰ are selected from halogen, CF₃, and NO₂,
Q is selected from a bond, optionally substituted alkylene, optionally substituted alkylene-C(O), optionally substituted phenylene, optionally substituted phenylene-C(O), optionally substituted cycloalkylene, optionally substituted cycloalkylene-C(O), optionally substituted alkyl cycloalkylene, optionally substituted alkylcycloalkylene-C(O), optionally substituted cycloalkylenealkyl, and optionally substituted cycloalkylenealkyl-C(O),
R¹ is selected from H, optionally substituted alkyl, optionally substituted aryl, optionally substituted heteroaryl, optionally substituted heteroalkyl, and optionally substituted heterocycloalkyl, and
R² is selected from H, hydroxyl, amino, optionally substituted alkyl, optionally substituted aryl, optionally substituted arylalkyl, optionally substituted alkylamine, optionally substituted arylamine, optionally substituted alkoxy, optionally substituted acyl, optionally substituted aminoacyl, optionally substituted alkoxyacyl, optionally substituted alkylthioacyl, optionally substituted arylaminoacyl, optionally substituted aryloxyacyl, optionally substituted arylthioacyl, optionally substituted heteroaryl, optionally substituted heteroalkyl, optionally substituted heterocycloalkyl and optionally substituted acylamino, or, alternatively,
R¹ and R² are optionally linked together to form an optionally substituted ring of up to about seven atoms including the N to which both are attached,
or a pharmaceutically acceptable salt thereof,
with the proviso that when L¹ is NR¹³ and Q is alkylene, L² is not hydrogen.

2. The compound or salt of claim 1, wherein R⁵, R⁶, R⁷, and R⁸ are independently selected from hydrogen, halogen, CF₃, optionally substituted alkyl, optionally substituted alkoxy, optionally substituted aryl, NO₂, optionally substituted acylamino, optionally substituted arylamino, optionally substituted acyl, optionally substituted acyloxy, or any of R⁵ and R⁶ taken together, R⁶ and R⁷ taken together, or R⁷ and R⁸ taken together form a 5- or 6-membered saturated or unsaturated ring.

3.-4. (canceled)

5. The compound or salt of claim 1, wherein R⁹, R¹⁰, R¹¹, and R¹² are independently selected from halogen, CF₃, optionally substituted alkyl, optionally substituted alkoxy, optionally substituted aryl, NO₂, optionally substituted acylamino, optionally substituted arylamino, optionally substituted acyl, optionally substituted acyloxy, or any of R⁵ and R⁶ taken together, R⁶ and R⁷ taken together, or R⁷ and R⁸ taken together form a 5- or 6-membered saturated or unsaturated ring.

6.-7. (canceled)

8. The compound or salt of claim 1, wherein R¹³ is selected from hydrogen, optionally substituted acyl, optionally substituted acyloxy, optionally substituted alkoxyacyl, optionally substituted aryloxyacyl, optionally substituted alkyl, optionally substituted aryl, optionally substituted arylalkyl, optionally substituted cycloalkyl, and optionally substituted cycloalkylalkyl.

9. (canceled)

10. The compound or salt of claim 1, wherein R¹⁴ is hydrogen or optionally substituted alkyl.

11. (canceled)

12. The compound or salt of claim 1, wherein R¹⁵, R¹⁶, R¹⁷, and R¹⁸ are independently selected from H and optionally substituted alkyl.

13. (canceled)

14. The compound or salt of claim 1, wherein Q is selected from a bond, optionally substituted alkylene-C(O), optionally substituted phenylene-C(O), optionally substituted cycloalkylene-C(O), optionally substituted alkylcycloalkylene-C(O), and optionally substituted cycloalkylenealkyl-C(O).

15. The compound or salt of claim 14, wherein Q is optionally substituted alkylene-C(O).

16. The compound or salt of claim 1, wherein R¹ is selected from H, optionally substituted alkyl, and optionally substituted aryl, and wherein R² is selected from H, optionally substituted alkyl, optionally substituted aryl, and optionally substituted arylalkyl, or, alternatively, R¹ and R² are optionally linked together to form an optionally substituted ring of up to about seven atoms including the N to which both are attached.

17. (canceled)

18. The compound or salt of claim 1, wherein J is J¹.

19.-33. (canceled)

34. The compound or salt of claim 1, wherein the compound is

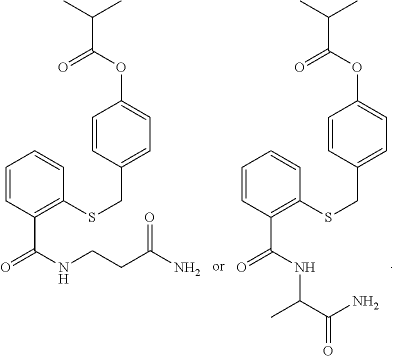 [SMILES: CC(C)C(=O)Oc1ccc(CSc2ccccc2C(=O)NCCC(N)=O)cc1 ; "or" ; CC(C)C(=O)Oc1ccc(CSc2ccccc2C(=O)NC(C)C(N)=O)cc1 ; "."]

35. (canceled)

36. The compound or salt of claim 1, wherein the compound is selected from:

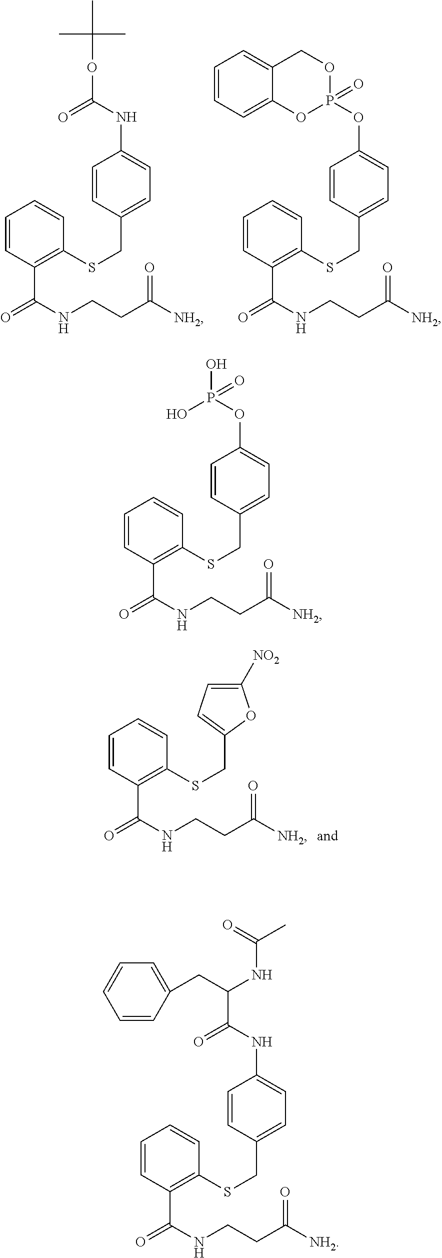

37. A pharmaceutical composition comprising a compound of claim 1 or a pharmaceutically acceptable salt thereof, and a pharmaceutically acceptable carrier.

38.-39. (canceled)

40. A composition comprising a compound of claim 1 or a pharmaceutically acceptable salt thereof and further comprising blood plasma, nutrient media, protein, a pharmaceutical, a cosmetic, a sperm or oocyte preparation, cells, cell cultures, bacteria, viruses, food or drink.

41. A method for dissociating a metal ion from a zinc finger-containing protein, the method comprising contacting said zinc finger-containing protein with a compound of claim 1 and pharmaceutically acceptable salts thereof.

42.-45. (canceled)

46. The method of claim 41, wherein the protein is that of a retrovirus selected from an HIV-1, an HIV-2, an SIV, a BIV, an EIAV, a Visna, a CaEV, an HTLV-1, a BLV, an MPMV, an MMTV, an RSV, an MuLV, a FeLV, a BaEV and an SSV retrovirus.

47. (canceled)

48. The method of claim 41, wherein the zinc finger-containing protein is that of a virus selected from the group consisting of an avian sarcoma retroviral group, a mammalian B-type retroviral group, a human T cell leukemia retroviral group, a bovine leukemia retroviral group, a D-type retroviral group, a murine leukemia-related group and a lentivirus group.

49.-51. (canceled)

52. A method for inactivating a virus, the method comprising contacting a virus with a compound of claim 1 or a pharmaceutically acceptable salt thereof, whereby contacting the virus with said compound or salt inactivates the virus.

53. The method of claim 52 wherein the virus is selected from an HIV-1, an HIV-2, an SIV, a BIV, an EIAV, a Visna, a CaEV, an HTLV-1, a BLV, an MPMV, an MMTV, an RSV, an MuLV, a FeLV, a BaEV and an SSV retrovirus.

54.-66. (canceled)

67. A method for inhibiting the transmission of a virus, the method comprising contacting a virus with a compound of claim 1 or a pharmaceutically acceptable salt thereof, whereby contacting the virus with said compound or salt inhibits the transmission thereof.

68. The method of claim 67, wherein the virus is selected from an HIV-1, an HIV-2, an SIV, a BIV, an EIAV, a Visna, a CaEV, an HTLV-1, a BLV, an MPMV, an MMTV, an RSV, an MuLV, a FeLV, a BaEV and an SSV retrovirus.

69.-85. (canceled)
